(19) United States
(12) Patent Application Publication
(10) Pub. No.: US 2014/0163448 A1
(43) Pub. Date: Jun. 12, 2014

(54) SUPRACHORIODAL DRAINAGE TUBE IN A FLOW CONTROL SYSTEM

(71) Applicant: ALCON RESEARCH, LTD., FORT WORTH, TX (US)

(72) Inventors: CASEY JEAN LIND, ORANGE, CA (US); ROBERT JOSEPH SANCHEZ, JR., OCEANSIDE, CA (US); ROBERT M. FELDMAN, HOUSTON, TX (US)

(73) Assignee: ALCON RESEARCH, LTD., FORT WORTH, TX (US)

(21) Appl. No.: 14/062,290

(22) Filed: Oct. 24, 2013

Related U.S. Application Data
(60) Provisional application No. 61/735,160, filed on Dec. 10, 2012.

(51) Int. Cl. A61F 9/007

(52) U.S. Cl. CPC A61F 9/00781
USPC 604/9; 604/8

(57) ABSTRACT

Described herein is a treatment device for the drainage of fluid within an eye of a patient. The treatment device comprises a drainage tube and a flow system in fluid communication with the drainage tube. The drainage tube has a lumen and comprises an inlet tube portion and an outlet tube portion, and is configured to convey aqueous humor through the lumen from an anterior chamber of the eye to a suprachoroidal space of the eye. The inlet tube portion extends from the anterior chamber to the flow system, and the outlet tube portion is flexible to conform to the curvature of the suprachoroidal space and extends from the valve system to the suprachoroidal space. The flow system is configured to control intraocular pressure by throttling flow rates of the aqueous humor through the drainage tube.

### BACKGROUND

The present disclosure relates generally to pressure/flow control systems and methods for use in treating a medical condition. In some instances, embodiments of the present disclosure are configured to be part of an IOP control system for the treatment of ophthalmic conditions.

Glaucoma, a group of eye diseases affecting the retina and optic nerve, is one of the leading causes of blindness worldwide. Most forms of glaucoma result when the intraocular pressure (IOP) increases to pressures above normal for prolonged periods of time. IOP can increase due to high resistance to the drainage of the aqueous humor relative to its production. Left untreated, an elevated IOP causes irreversible damage to the optic nerve and retinal fibers resulting in a progressive, permanent loss of vision.

The eye's ciliary body continuously produces aqueous humor, the clear fluid that fills the anterior segment of the eye (the space between the cornea and lens). The aqueous humor flows out of the anterior chamber (the space between the cornea and iris) through the canalicular and the uveoscleral pathways, both of which contribute to the aqueous drainage system. The delicate balance between the production and drainage of aqueous humor determines the eye's IOP.

FIG. 1 is a diagram of the front portion of an eye 10 that helps to explain the processes of glaucoma. In FIG. 1, representations of the lens 110, cornea 120, iris 130, ciliary body 140, trabecular meshwork 150, Schlemm's canal 160, the anterior chamber 170, the posterior chamber 175, the sclera 180, the retina 182, the choroid 185, the limbus 190, the suspensory ligaments or zonules 195, the suprachoroidal space 200, and the conjunctiva 202 are pictured. Aqueous fluid is produced by the ciliary body 140, which lies beneath the iris 130 and adjacent to the lens 110 in the anterior chamber 170 of the anterior segment of the eye. This aqueous humor washes over the lens 110 and iris 130 and flows to the drainage system located in the angle of the anterior chamber 170.

After production by the ciliary body 140, the aqueous humor may leave the eye by several different routes. Some goes posteriorly through the vitreous body behind the lens 110 to the retina, while most circulates in the anterior segment of the eye to nourish avascular structures such as the lens 110 and the cornea 120 before outflowing by two major routes: the canalicular route 205 and the uveosceral route 210. The angle of the anterior chamber 170, which extends circumferentially around the eye, contains structures that allow the aqueous humor to drain. The canalicular (or trabecular) route is the main mechanism of outflow, accounting for a large percentage of aqueous egress. The route extends from the anterior chamber angle (formed by the iris 130 and the cornea 120), through the trabecular meshwork 150, into Sclemm's canal 160. The trabecular meshwork 150, which extends circumferentially around the anterior chamber 170, is commonly implicated in glaucoma. The trabecular meshwork 150 seems to act as a filter, limiting the outflow of aqueous humor and providing a back pressure that directly relates to IOP. Schlemm's canal 160 is located just peripheral to the trabecular meshwork 150. Schlemm's canal 160 is fluidically coupled to collector channels (not shown) allowing aqueous humor to flow out of the anterior chamber 170. The arrows 205 show the flow of aqueous humor from the ciliary bodies 140, over the lens 110, over the iris 130, through the trabecular meshwork 150, and into Schlemm's canal 160 and its collector channels (to eventually reunite with the bloodstream in the episcleral vessels (not shown)).

The uveosceral route 210 accounts for the major remainder of aqueous egress in a normal eye, and also begins in the anterior chamber angle. Though the anatomy of the uveoscleral route 210 is less clear, aqueous is likely absorbed by portions of the peripheral iris 130, and the ciliary body 140, after which it passes into the suprachoroidal space 200. As shown in FIG. 2a, the suprachoroidal space 200 is a potential space of loose connective tissue between the sclera 180 and the choroid 185 that provides a pathway for uveoscleral outflow. Normally the suprachoroidal space is not evident due to the close apposition of the choroid 185 to the sclera 180 from the intraocular pressure of the eye. As shown in FIG. 2b, however, the tissues separate to form the suprachoroidal space 200 when fluid accumulates between the tissues. Aqueous exits the eye along the length of the suprachoroidal space to eventually reunite with the bloodstream in the episcleral vessels.

One method of treating glaucoma includes implanting a drainage device in a patient's eye. The drainage device allows fluid to flow from the interior chamber of the eye to a drainage site, relieving pressure in the eye and thus lowering IOP. Drainage devices that drain into the subconjunctival space require that a functional subconjunctival bleb be maintained to allow aqueous humor to be absorbed and drained away. However, subconjunctival blebs are associated with several complications, including bleb failure due to fibrosis, conjunctival leakage, infections, and/or endophthalmitis.

The system and methods disclosed herein overcome one or more of the deficiencies of the prior art.

### SUMMARY

In an exemplary aspect, the present disclosure is directed to a treatment device for the drainage of fluid within an eye of a patient, the treatment device includes a drainage tube having a lumen and comprising an inlet tube portion and an outlet tube portion. The drainage tube may be configured to convey aqueous humor through the lumen from an anterior chamber of the eye to a suprachoroidal space of the eye. The treatment device also includes a flow system in fluid communication with the drainage tube. The flow system may be configured to control intraocular pressure by throttling flow rates of the aqueous humor through the drainage tube. The inlet tube portion may be arranged to extend from the anterior chamber to the flow system and the outlet tube portion may be flexible to conform to the curvature of the suprachoroidal space and may be arranged to extend from the valve system to the suprachoroidal space.

In one aspect, the flow system is sized to be positioned within the subconjunctival space. In one aspect, the outlet tube portion includes a proximal end coupled to the flow system and a distal end arranged to be positioned within the suprachoroidal space and includes an outlet tube lumen extending from the proximal end to the distal end.

In another exemplary aspect, the present disclosure is directed to a treatment device for the drainage of fluid within an eye of a patient. The treatment device includes an implant positionable in a subconjunctival space of the eye and includes an outlet tube portion having a lumen extending from a proximal aperture to a distal aperture. The outlet tube portion may be configured to convey aqueous humor through the lumen from the implant to a suprachoroidal space of the eye. The outlet tube portion may be arranged to extend from the implant through a sclera of the eye into the anterior chamber and may be arranged to curve back on itself to enter the suprachoroidal space. The proximal aperture is in fluid communication with the implant, and the distal aperture is arranged to be in communication with the suprachoroidal space.

In one aspect, the outlet tube portion is arranged to extend from the implant through a corneoscleral limbus, enter the anterior chamber, and curve back on itself to enter the suprachoroidal space. In one aspect, the outlet tube portion is configured to extend from the implant through a sclera. In one aspect, the outlet tube portion is arranged to extends from the implant through the sclera and curve back on itself within the sclera to enter the suprachoroidal space.

In another exemplary aspect, the present disclosure is directed to a method of implanting a treatment device into an eye of a patient. The method includes inserting a drainage device including a flow system, an inlet tube, and an outlet tube into a subconjunctival space. The inlet tube includes a first proximal end coupled to the flow system in the subconjunctival space. The outlet tube includes a second proximal end coupled to the flow system in the subconjunctival space. The method also includes passing a first distal end of the inlet tube through a sclera into an anterior chamber and includes passing a second distal end of the outlet tube through the sclera into the suprachoroidal space.

In one aspect, passing a second distal end of the outlet tube through the sclera into the suprachoroidal space comprises turning the outlet tube back on itself within the sclera before the second distal end enters the suprachoroidal space. In one aspect, passing a second distal end of the outlet tube through the sclera into the suprachoroidal space comprises passing the second distal end of the outlet tube into the anterior chamber and turning the outlet tube back on itself within the anterior chamber before the second distal end enters the suprachoroidal space. In one aspect, passing a second distal end of the outlet tube through the sclera into the suprachoroidal space comprises passing the second distal end of the outlet tube into the anterior chamber and turning the outlet tube back on itself within the anterior chamber to pass adjacent to a scleral spur before entering the suprachoroidal space.

It is to be understood that both the foregoing general description and the following detailed description are exemplary and explanatory in nature and are intended to provide an understanding of the present disclosure without limiting the scope of the present disclosure. In that regard, additional aspects, features, and advantages of the present disclosure will be apparent to one skilled in the art from the following detailed description.

### BRIEF DESCRIPTION OF THE DRAWINGS

The accompanying drawings illustrate embodiments of the devices and methods disclosed herein and together with the description, serve to explain the principles of the present disclosure.

FIG. 1 is a diagram of the front portion of an eye.

FIGS. 2a and 2b are illustrations of a cross-sectional view of the suprachoroidal space and other associated ocular tissues shown in FIG. 1 (through lines 2-2).

FIG. 3 is a schematic diagram of an exemplary drainage device disposed on an eye according to the principles of the present disclosure.

FIG. 4 illustrates a cross-sectional side view of an exemplary drainage implant positioned within an eye according to one embodiment of the present disclosure.

FIGS. 5-10 illustrate perspective views of various exemplary outlet tubes according to the principles of the present disclosure.

FIG. 11 illustrates a partially cross-sectional perspective view of an exemplary drainage implant positioned within an eye according to one embodiment of the present disclosure.

FIG. 12 illustrates a cross-sectional side view of an exemplary drainage implant positioned within an eye according to one embodiment of the present disclosure.

FIG. 13 illustrates an exemplary tunneling instrument inserted into the anterior chamber of an eye according to one embodiment of the present disclosure.

FIG. 14 illustrates the exemplary tunneling instrument shown in FIG. 13 being inserted into a suprachoroidal space according to the principles of the present disclosure.

FIG. 15 illustrates an exemplary guiding instrument manipulating an exemplary outlet tube within the anterior chamber of an eye according to one embodiment of the present disclosure.

FIG. 16 illustrates an exemplary outlet tube positioned within the suprachoroidal space according to one embodiment of the present disclosure.

FIG. 17 illustrates a partially cross-sectional perspective view of an exemplary drainage implant positioned within an eye according to one embodiment of the present disclosure.

FIG. 18 illustrates an exemplary tunneling instrument inserted into through the sclera and into the suprachoroidal space of an eye according to one embodiment of the present disclosure.

FIG. 19 illustrates an exemplary outlet tube positioned through the sclera and within the suprachoroidal space according to one embodiment of the present disclosure.

FIG. 20 illustrates an exemplary outlet tube positioned through the sclera and within the suprachoroidal space according to one embodiment of the present disclosure.

### DETAILED DESCRIPTION

For the purposes of promoting an understanding of the principles of the present disclosure, reference will now be made to the embodiments illustrated in the drawings, and specific language will be used to describe the same. It will nevertheless be understood that no limitation of the scope of the disclosure is intended. Any alterations and further modifications to the described devices, instruments, methods, and any further application of the principles of the present disclosure are fully contemplated as would normally occur to one skilled in the art to which the disclosure relates. In particular, it is fully contemplated that the features, components, and/or steps described with respect to one embodiment may be combined with the features, components, and/or steps described with respect to other embodiments of the present disclosure. For the sake of brevity, however, the numerous iterations of these combinations will not be described separately. For simplicity, in some instances the same reference numbers are used throughout the drawings to refer to the same or like parts.

The present disclosure is directed to drainage from a flow control system for treating a medical condition, such as glaucoma. In one aspect, the system adjusts IOP by regulating fluid drainage through an implant such as a glaucoma drainage device (GDD). The system directs fluid drainage from the anterior chamber of an eye through a drainage tube to a drainage site remote from the subconjunctiva. In one aspect, the system directs fluid drainage to the suprachoroidal space through the drainage tube. In one aspect, the flow control system is implanted in the subconjuctival space, and an outlet tube portion of the drainage tube travels from the flow control system directly through the sclera to drain fluid into the suprachoroidal space. In another aspect, the flow control system is implanted in the subconjuctival space, and the outlet tube travels from the flow control system through the sclera into the anterior chamber before draining fluid into the suprachoroidal space. Thus, the devices, systems, and methods disclosed herein allow for the flow control system to reside within the subconjunctival space while providing an outlet tube to facilitate draining the aqueous humor away into the suprachoroidal space, thereby allowing for a subconjunctival atmospheric pressure reference in conjunction with bleb-free drainage.

FIG. 3 is a schematic diagram of an exemplary drainage device or implant 300 positioned within an eye of a patient. The drainage implant 300 is designed to regulate IOP by utilizing an adjustable smart valve, passive valve, or active element (e.g., without limitation, a pump) to throttle or pump the flow of aqueous humor out of the aqueous chamber into the suprachoroidal space.

In this example, the implant 300 includes a drainage tube 305 and a divider 310 associated with a flow system 315. In some examples, the flow system 315 may be formed as a part of or utilized in a valve system such as those disclosed in application Ser. No. 13/315,329, titled “Active Drainage Systems with Pressure-Driven Valves and Electronically-Driven Pump,” incorporated herein by reference.

In the embodiment pictured in FIG. 3, the implant 300 is arranged in the eye such that three areas of pressure interact with the implant: P1, P2, and P3. Pressure area P1 reflects the pressure of the anterior chamber 170, pressure area P2 reflects the pressure of a drainage site 320, and pressure area P3 reflects a pressure located remotely from P1 and P2 (effectively reflecting atmospheric pressure). In some embodiments, pressure area P1 reflects the pressure located in a lumen or tube that is in fluidic communication with the anterior chamber 170.

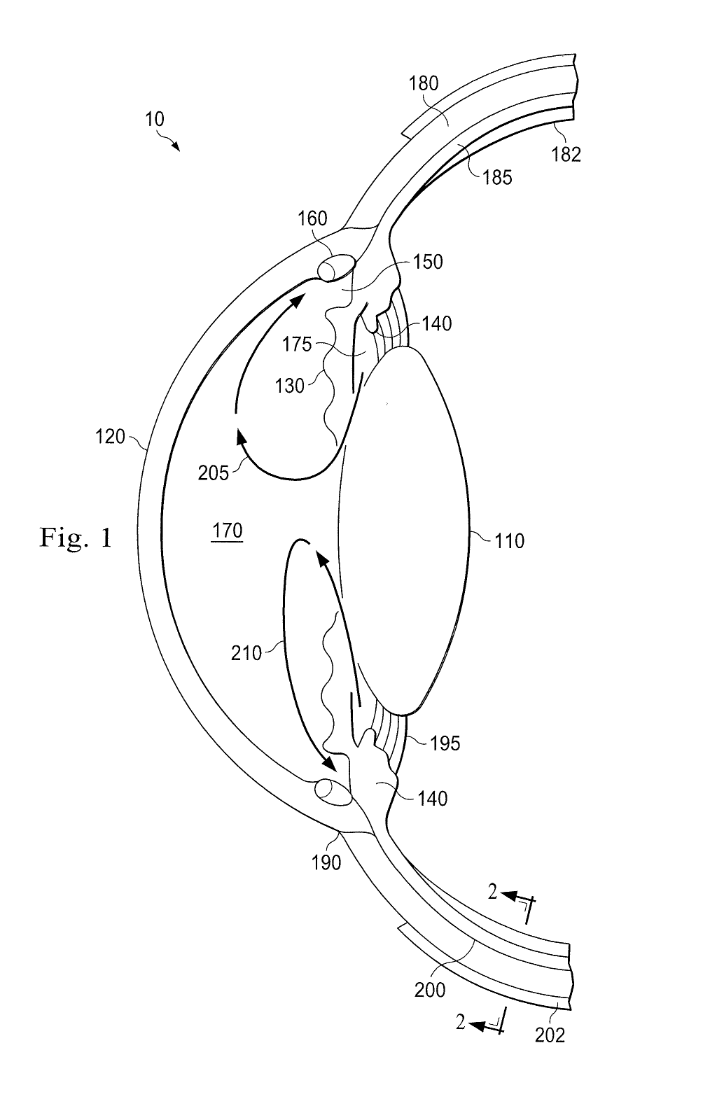

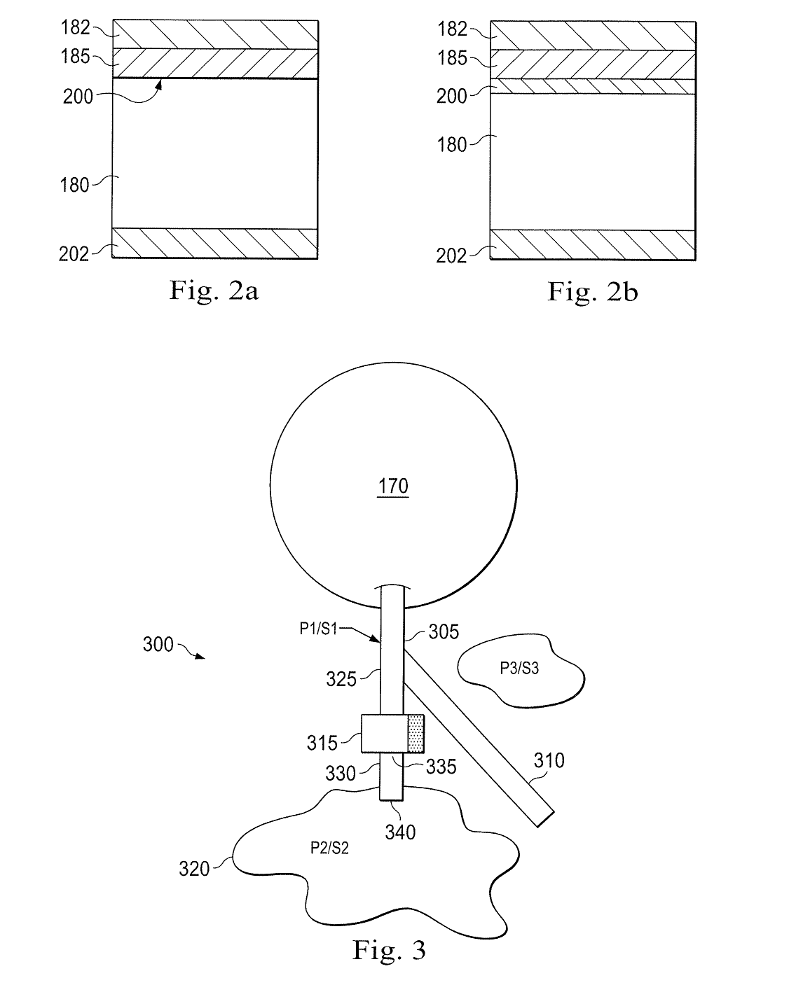

The drainage tube 305 drains aqueous humor from the anterior chamber 170 of the eye to the drainage location 320, which may be the suprachoroidal space 200 (shown in FIGS. 1-2b). Other examples of a drainage location 320 include, but are not limited to: a subscleral space, a supraciliary space, an episcleral vein, and other uveo-scleral pathways. The drainage tube 305 includes an inlet tube or inlet tube portion 325, which extends from the anterior chamber 170 to the flow system 315, and an outlet tube or outlet tube portion 330, which extends from the flow system 315 to the drainage site 320. The outlet tube 330 includes a proximal end 335 coupled to the flow system 315 and a distal end 340 positioned within the drainage site 320.

The flow system 315 regulates IOP by throttling or inducing the flow of aqueous humor through the tube 305, from the inlet tube 325 to the outlet tube 330. In some instances, the flow system 315 throttles the flow of aqueous humor through the tube 305 as a function of a pressure differential. The flow system 315 may include components or elements that control pressure by regulating the amount of drainage flow through the implant 300. The flow system 315 may include any number of valves and any number of pumps, or may not include a pump or may not include a valve. In some embodiments, the flow system 315 is an active system that is responsive to signals from a processor to increase flow, decrease flow, or to maintain a steady flow as a function of pressure differentials across the valve system. In one embodiment, it does this by maintaining a valve setting at a consistent setting, or increasing or decreasing the amount that the valve is open.

In addition, the flow system 315 may incorporate pressure sensors to monitor and utilize the pressures P1, P2, and P3 to achieve a desired IOP. In some embodiments, the implant 300 responds to the pressure differentials between the pressures sensed at P1, P2, and P3 by sensors S1, S2, and S3, respectively, to control the flow system 315 and thereby throttle the flow rate of aqueous humor through the drainage tube 305 to control IOP. In some embodiments, the various pressure differentials across the pressure areas sensed at P1, P2, and P3 (P1−P2, P1−P3, P2−P3) drive the flow system 315 and dictate the valve position or pump state to throttle the flow rate of aqueous humor through the drainage tube 305 to control IOP.

In the embodiment shown, a pressure sensor S1 measures the pressure in the tube 305 upstream from the flow system 315 and downstream from the anterior chamber 170. In this manner, the pressure sensor S1 measures the pressure in the anterior chamber 170. The expected measurement discrepancy between the true anterior chamber pressure and that measured by S1 when located in a tube downstream of the anterior chamber (even when located between the sclera and the conjunctiva) is negligible.

A pressure sensor S2 is located at the drainage site 320 or in fluid communication with the drainage site 320 via the outlet tube 320. As such, the pressure sensor S2 may be located in the suprachoroidal space 200, a subscleral space, a supraciliary space, an episcleral vein, or another uveo-scleral pathway, for example.

In some embodiments, the divider 310 acts as a barrier that separates the pressure region measured by the pressure sensor S3 from the pressure region measured by the pressure sensor S2. In some embodiments, the system includes other barriers that separate the sensors S1, S2, and S3. These barriers may be elements of the flow system 315 itself. In FIG. 3, the pressure region measured by the pressure sensor S3 is physically separated from the pressure region measured by the pressure sensor S2 by the divider 310. The divider 310 is a physical structure that separates the drainage area 306 from the isolated location of pressure region measured by the pressure sensor S3.

Generally, IOP is a gauge pressure reading—the difference between the absolute pressure in the eye (as measured by sensor S1) and atmospheric pressure (as measured by sensor S3). Atmospheric pressure, typically about 760 mm Hg, often varies in magnitude by 10 mmHg or more depending on weather conditions or indoor climate control systems. In addition, the effective atmospheric pressure can vary significantly—in excess of 300 mmHg—if a patient goes swimming, hiking, riding in an airplane, etc. Such a variation in atmospheric pressure is significant since IOP is typically in the range of about 15 mm Hg. Thus, for accurate monitoring of IOP, it is desirable to have pressure readings for the anterior chamber (as measured by sensor S1) and atmospheric pressure in the vicinity of the eye (as measured by sensor S3).

In one embodiment of the present invention, pressure readings are taken by the pressure sensors S1 and S3 simultaneously or nearly simultaneously over time so that the actual IOP can be calculated (as S1−S3 or S1−f(S3), where f(S3) indicates a function of S3). In another embodiment of the present invention, pressure readings taken by the pressure sensors S1, S2, and S3 can be used to control a device that drains aqueous from the anterior chamber 170. For example, in some instances, the implant 300 reacts to the pressure differential across S1 and S3 continuously or nearly continuously so that the actual IOP (as P1−S3 or P1−f(S3)) can be responded to accordingly.

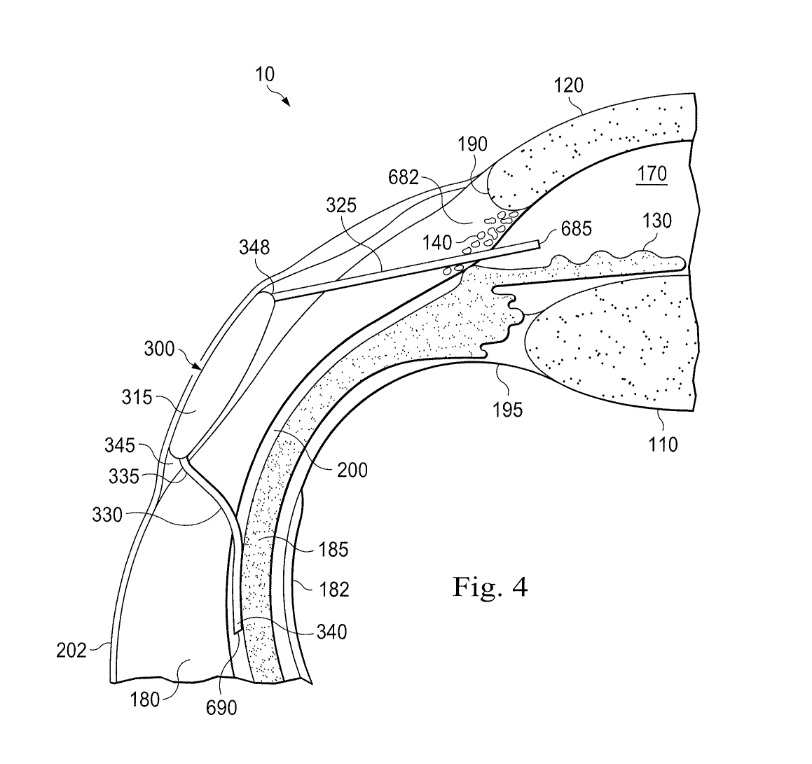

As shown in FIG. 4, the drainage implant 300 is shaped and configured to be implanted within the subconjunctival space, between the conjunctiva 202 and the sclera 180 (shown in FIGS. 1-2b). In some embodiments, the bulk of the implant 300 may be positioned within the eye in a subconjunctival space 345 between the conjunctiva 202 and the sclera 180 with an anterior border 348 of the flow system 315 positioned such that the implant does not come into contact with the optic nerve (pending the size and shape of the implant, approximately 8 to 10 mm posterior to the limbus 190 (the border between the cornea and the sclera). The drainage implant 300 may be held in place within the eye via anchoring sutures, the angle of implantation and surrounding anatomy, or by a spring force or other mechanisms that stabilize the implant 300 relative to the patient's eye. The inlet tube 325 and the outlet tube 330 are coupled to the flow system 315 at the location of the subconjunctival space 345, and extend from the subconjuctival space 345 into the anterior chamber 170 and the suprachoroidal space 200, respectively, as discussed below.

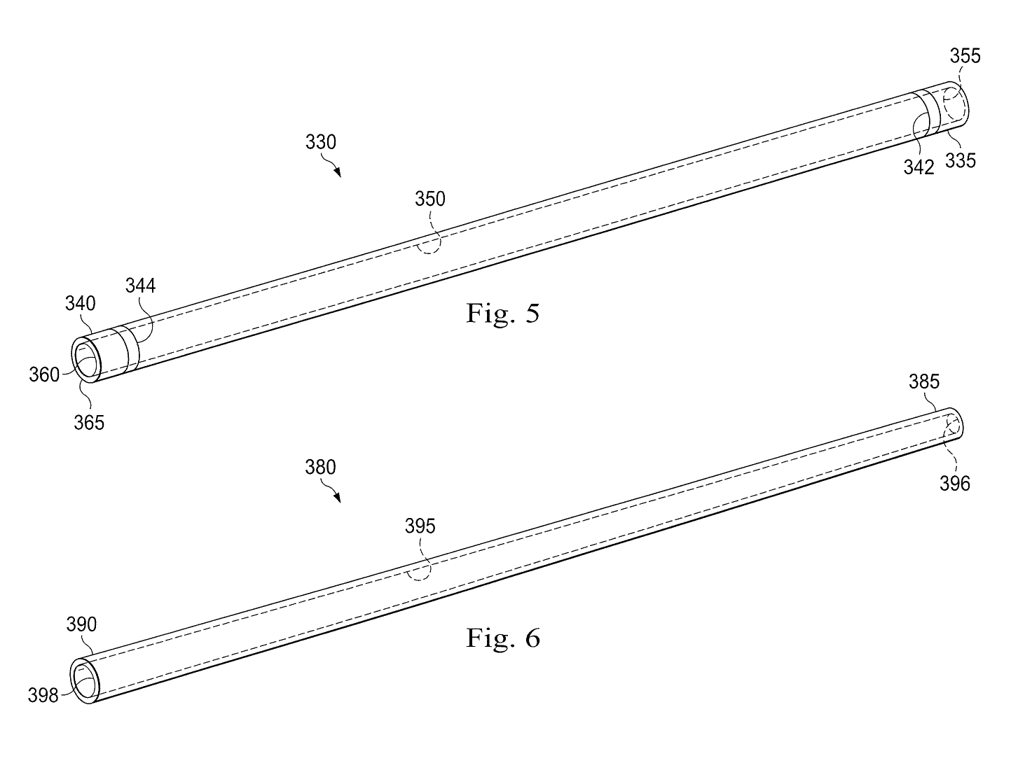

FIG. 5 illustrates the exemplary outlet tube 330 shown in FIGS. 3 and 4. The outlet tube 330 is shaped and configured as an elongate, flexible, hollow cylinder including the proximal end 335, the distal end 340, a lumen 350 extending from the proximal end 335 to the distal end 340, a proximal marker 342, and a distal marker 344. In other embodiments, the outlet tube may be of any of a variety of different shapes. The proximal end 335 is coupled to the flow system 315 (as shown in FIG. 4), and is configured to receive aqueous humor from the flow system 315. The distal end 340 is configured to allow the egress of aqueous humor into the suprachoroidal space. The lumen 350 serves as a passageway for the flow of aqueous humor through the outlet tube 330 from the flow system 315 into the drainage site 320 shown in FIG. 3 (e.g., the suprachoroidal space). In the pictured embodiment, the lumen 350 has a uniform luminal diameter along the length of the tube 330. In other embodiments, the luminal diameter can vary in diameter along the length of the tube. For example, in some embodiments, the luminal diameter may taper along the length of the tube so as to achieve a desired flow rate through the tube.

The outlet tube 330 includes a single proximal aperture 355 at the proximal end 335 for ingress of fluid, and a single distal aperture 360 for egress of fluid. Both apertures 355, 360 are in communication with the lumen 350. However, other embodiments may include any number and arrangement of apertures that communicate with the lumen 350, as discussed below. In one embodiment, aqueous humor can flow from the flow system 315 into the proximal aperture 355 at the proximal end 335 of the outlet tube 330, through the lumen 350, and out the distal aperture 360 at the distal end 340 into the suprachoroidal space.

In the pictured embodiment, the outlet tube 330 has an atraumatic distal end 340, shaped and configured with blunt edges 365 to prevent inadvertent injury to ocular tissues (e.g., the spongy choroid) during implantation or if the tube 330 moves after implantation. In some embodiments, the edges 365 may be shaped in an atraumatic manner, such as by having a rounded profile. In some embodiments, the edges 365 may be manufactured of or be coated with a soft material. In other embodiments, the distal end 340 may be shaped and configured to permit the outlet tube to pierce ocular tissue and enter the suprachoroidal space without the assistance of a delivery device or a pre-created pathway into the suprachoridal space. For example, in some embodiments, the edges 365 may be sufficiently sharp to cut through ocular tissues.

In some embodiments, the distal end 340 has a column strength sufficient to permit the outlet tube 330 to be inserted into the suprachoroidal space such that the distal aperture 360 tunnels through the ocular tissue without structural collapse or degradation of the tube 330. In some embodiments, the column strength is sufficient to permit the tube 330 to tunnel through ocular tissues into the suprachoridal space without any structural support from an additional structural such as a delivery device. In other embodiments, a delivery device may be used to facilitate the progress of the outlet tube 330 through the ocular tissue toward the suprachoroidal space.

The outlet tube 330 may include one or more features that aid in properly positioning the tube in the eye. For example, the markers 342, 344 comprise positional indicators that can be used to accurately position the tube 330 in the eye. The marker 342 is positioned adjacent the proximal end 335 of the tube 330, and the marker 344 is positioned adjacent the distal end 340 of the tube 330. In other embodiments, the tube 330 may include any number and arrangement of markers. The markers 342, 344 may comprise visual, tomographic, echogenic, or radiopaque markers. In one exemplary method of using the markers to properly position the outlet tube 330, the distal end 340 of the outlet tube 330 may be inserted into the suprachoroidal space until either the marker 344 or the marker 342 is aligned with an appropriate anatomic structure or surgical indicator (e.g., a suture). For example, the a surgeon may advance the distal end 340 into the suprachoroidal space until the marker 342 aligns with an appropriate anatomic structure, such as, by way of non-limiting example, the scleral spur, the limbus, or the trabecular meshwork, thereby indicating that an adequate length of the tube 330 has entered the suprachoroidal space.

The outlet tube 330 has a substantially uniform diameter along its entire length. In exemplary embodiments, the outer diameter of the outlet tube may range in size from about 0.010 in (0.254 mm) to 0.040 in (1.016 mm). In one embodiment, the outer diameter of the outlet tube 330 may be 0.025 in (0.635 mm). However, this disclosure supports outlet tubes of different shapes and dimensions, and outlet tubes of the present disclosure may be of any shape and any dimension that may be accommodated by the eye, and in particular the suprachoroidal space.

Although the outlet tube 330 is shown having a circular cross-sectional shape, the outlet tube may have any of a variety of cross-sectional shapes, including without limitation, an ovoid, elliptical, square, rhomboid, or rectangular shape. In some embodiments, the outlet tube may vary in cross-sectional shape along its length. The particular cross-sectional shape may be selected to facilitate easy insertion into the eye, and may be dependent upon the method of insertion planned. In some embodiments, the outlet tube 330 may have a predetermined radius of curvature that conforms to the radius of curvature of the suprachoroidal space. In other embodiments, the outlet tube 330 may be sufficiently flexible to assume the radius of curvature of the suprachoroidal space after implantation within the space.

As mentioned above, the outlet tube 330 has a substantially uniform diameter along its entire length. In other embodiments, as shown in FIG. 6, the diameter of the outlet tube may vary along its length. FIG. 6 illustrates an exemplary outlet tube 380 including a proximal end 385 and a distal end 390 with a lumen 395 extending therebetween. The tube 380 is substantially similar to the outlet tube 330 except for the differences described herein. The outlet tube 380 includes a proximal aperture 396 and a distal aperture 398. The diameter of the outlet tube 380, and consequently the diameter of the lumen 395, tapers from the distal end 390 to the proximal end 385. The diameter increases from the proximal aperture 396 to the distal aperture 398. By having a variable inner diameter that gradually increases from the proximal end 385 to the distal end 390, a pressure gradient is produced that may help aqueous humor and/or particulate matter that may otherwise clog the tube to progress toward the distal aperture 398 and exit the tube. Other embodiments may have other configurations of varying diameter. The taper may exist along the entire length of the tube or may exist along only one or more portions of the tube (e.g., the distal portion). For example, in other embodiments, the outlet tube may taper from a proximal end to a distal end, or widen one in a middle portion of the tube. In exemplary embodiments, the inner diameter of the outlet tube (i.e., the diameter of the lumen) may range in size from about 0.005 in (0.127 mm) to 0.100 in (2.54 mm). In particular, the inner diameter of the outlet tube may range in size from about 0.005 in (0.127 mm) to 0.050 in (1.27 mm) at the proximal aperture, and may range in size from about 0.020 in (0.508 mm) to 0.100 in (2.54 mm) at the distal aperture. In one example, the inner diameter of the outlet tube may be 0.025 in (0.635 mm) at the proximal aperture, and may be 0.035 in (0.889 mm) at the distal aperture.

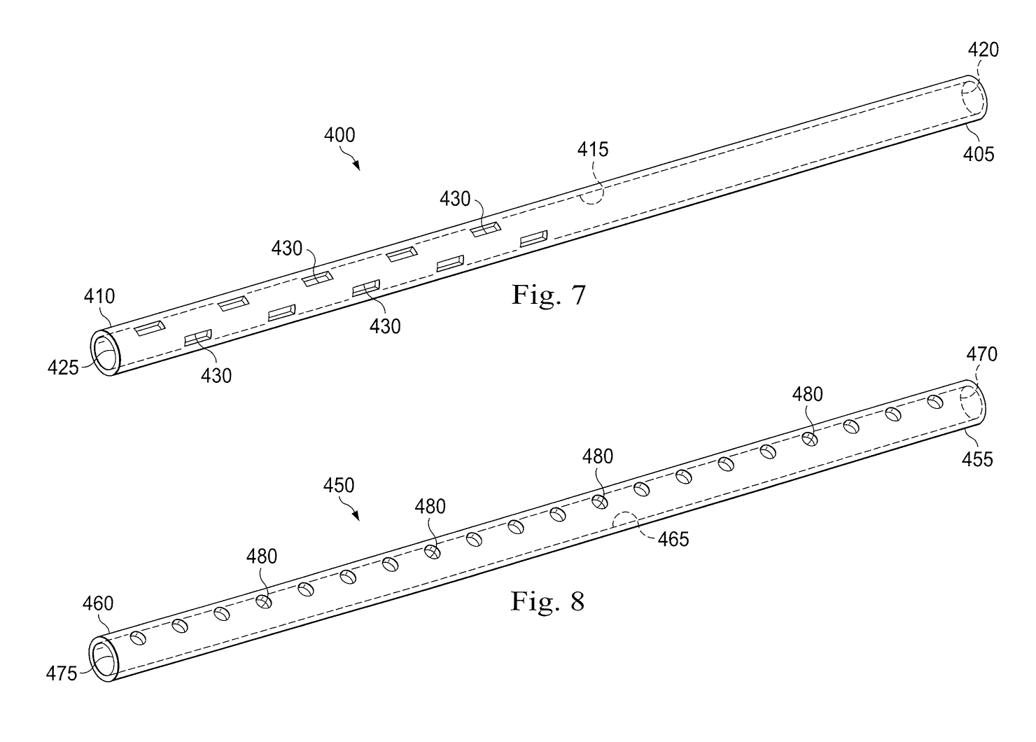

In the embodiment shown in FIG. 5, as mentioned above, the outlet tube 330 includes the single proximal aperture 355 at the proximal end 335 for ingress of fluid, and a single distal aperture 360 for egress of fluid. In other embodiments, as indicated in FIGS. 7 and 8, the outlet tube can include a plurality of apertures through which fluid may exit the tube. FIG. 7 illustrates an outlet tube 400 according to one embodiment of the present disclosure. The tube 400 is substantially similar to the outlet tube 330 except for the differences described herein. The tube 400 includes a proximal end 405, a distal end 410, and a lumen 415 extending from the proximal end 405 to the distal end 410. In addition to a proximal aperture 420 and a distal aperture 425, both of which are in communication with the lumen 415, the outlet tube 400 includes a plurality of holes 430 located along at the length of the outlet tube 400. The holes 430 are in fluid communication with the lumen 415, and allow fluid to exit the lumen 415 of the outlet tube 400 and enter the tissues or space surrounding the tube 400. In the pictured embodiment, the holes 430 are interspersed in a staggered pattern along the distal half of the tube 400, but, in other embodiments, the holes may be arranged in any of a variety of patterns, both asymmetrical and symmetrical, along any portion (or entirety) of the tube. In FIG. 7, the illustrated holes 400 are shaped as rectangular apertures, but, in other embodiments, the holes may have any of a variety of shapes, including, without limitation, circular, ovoid, rhomboid, and square. It should be noted that the spatial configuration, size, and angle of the holes may vary in different embodiments. Multiple apertures or holes in the tube guard against the blockage of flow through the tube in instances where other holes or apertures may be blocked. In some embodiments, the holes may function as visual markers to aid in positioning the outlet tube within the eye.

For example, FIG. 8 illustrates an outlet tube 450 according to another embodiment of the present disclosure. The tube 450 is substantially similar to the outlet tube 400 except for the differences described herein. The tube 450 includes a proximal end 455, a distal end 460, and a lumen 465 extending from the proximal end 455 to the distal end 460. In addition to a proximal aperture 470 and a distal aperture 475, both of which are in communication with the lumen 465, the outlet tube 450 includes a plurality of holes 480 located along at the entire length of the outlet tube 400. The holes 480 are in fluid communication with the lumen 465, and allow fluid to exit the lumen 465 of the outlet tube 450 and enter the tissues or space surrounding the tube 450. In FIG. 8, the illustrated holes 480 are spaced symmetrically along the entire length of the tube 450, and the holes 480 have a circular shape.

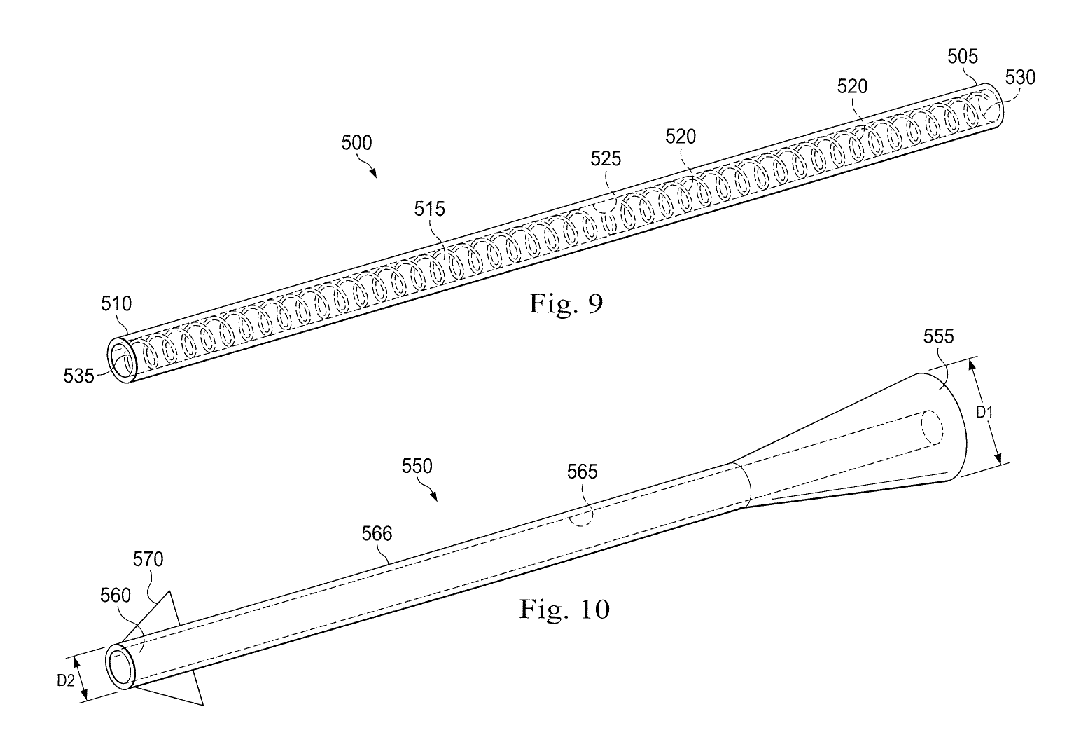

FIG. 9 illustrates an outlet tube 500 according to another embodiment of the present disclosure. The tube 500 is substantially similar to the outlet tube 330 except for the differences described herein. The tube 500 includes a proximal end 505, a distal end 510, and a lumen 515 extending from the proximal end 505 to the distal end 510. The outlet tube 500 includes a plurality of drainage features 520 located along an interior or luminal wall 525 of the tube 500. In the pictured embodiment, the drainage features 520 comprise rungs of a spiral shape extending from a proximal aperture 530 to a distal aperture 535. The drainage features 520 are shaped and arranged within the tube to facilitate the passage of fluid through the tube from the proximal aperture to the distal aperture. In FIG. 9 the illustrated spiral drainage features 520 are spaced symmetrically along the entire length of the tube 500. In other embodiments, the drainage features may be arranged symmetrically or asymmetrically, and may be arrayed along only a portion of the tube. In other embodiments, the drainage features 520 may comprise any of a variety of shapes, including, without limitation, protrusions such as nubs or ribs, indentations, dimples, columns, or helices.

FIG. 10 illustrates an outlet tube 550 according to another embodiment of the present disclosure. The tube 550 is substantially similar to the outlet tube 330 except for the differences described herein. The tube 550 includes a proximal end 555, a distal end 560, and a lumen 565 extending from the proximal end 555 to the distal end 560. The outlet tube 550 has a flared shape at the proximal end 555 that is designed to prevent the outlet tube from moving further into the suprachoroidal space after being properly positioned therein. In particular, the proximal end 555 has an outer diameter D1 that is wider than the outer diameter D2 of the distal end 560 (and the remainder of the outlet tube 550). The larger diameter D1 allows the proximal end 555 of the outlet tube 550 to lodge against tissue and prevents the outlet tube 550 from progressing further into the suprachoroidal space than desired. Although the proximal end 555 is relatively cone-shaped, the proximal end in other embodiments may have any of a variety of shapes designed to prevent the inadvertent progress of the outlet tube into a predetermined space such as the suprachoroidal space. In some embodiments, a midportion 566 of the tube 550 may have a larger diameter than the proximal end 555 and/or the distal end 560 of the tube to lodge against tissue and prevent the outlet tube from moving further into the suprachoroidal space after being properly positioned therein.

The outlet tube 550 includes retention features 570 that aid in anchoring the outlet tube 550 within the drainage site (e.g., the suprachoroidal space) after implantation of the outlet tube 550. In the pictured embodiment, the retention features 570 are shaped as wings that protrude from the exterior of the distal end 560 of the tube 550. In the pictured embodiment, the retention features 570 are shaped as triangular wings. In other embodiments, the retention features may comprise any of a variety of shapes, including without limitation, helical, rectangular, ovoid, cyclic, round, or combinations thereof. In other embodiments, the retention features may comprise any of a variety of structures, including without limitation, protrusions such as nubs, ribs, or prongs, textured surfaces, and indentations. The retention features 570 are configured to engage with the surrounding tissue and minimize inadvertent movement of the outlet tube after implantation. The retention features 570 may be flexible or stiff, or have varying degrees of flexibility. In some embodiments, the retention features 570 may include unexpanded and expanded conditions, and may be configured to transition from the unexpanded condition to the expanded condition after final positioning of the outlet tube. The retention features may be made of any of a variety of biocompatible materials, including, by way of non-limiting example, a polymer, Nitinol, or another shape memory material.

An outlet tube described herein may be flexible along its entire length, may have a predetermined stiffness along its entire length, or may have a varying degree of stiffness or flexibility along its entire length. Thus, the outlet tubes may be made from any of a variety of flexible, rigid, or composite materials. In particular, the outlet tubes described herein may be made from any of a variety of biocompatible materials having the requisite flexibility and hoop strength for adequate lumen support and drainage through the lumen after implantation. Such materials include, without limitation, silicone tubing, reinforced silicone tubing, PEEK, polycarbonate, or other flexible materials. In some instances, the tube may be scored or otherwise imprinted for added flexibility throughout the tube or only in one or more portions of the tube.

Any of the embodiments of the outer tube described herein may be coated on its inner luminal surface with one or more drugs or other materials designed to help maintain the patency of the lumen. Likewise, any of the embodiments of the outer tube described herein may be coated on its outer surface with one or more drugs or other materials designed to encourage healing and/or in-growth of ocular tissue around the outlet tube to assist in retention of the outlet tube (e.g., within the suprachoroidal space) or prevent an immune response to the outlet tube. Such drugs or other materials may be contained within a polymeric coating applied to the tube. Any of the embodiments of the outer tube described herein may be coated on its outer surface with a material that provides a contact surface to promote healing.

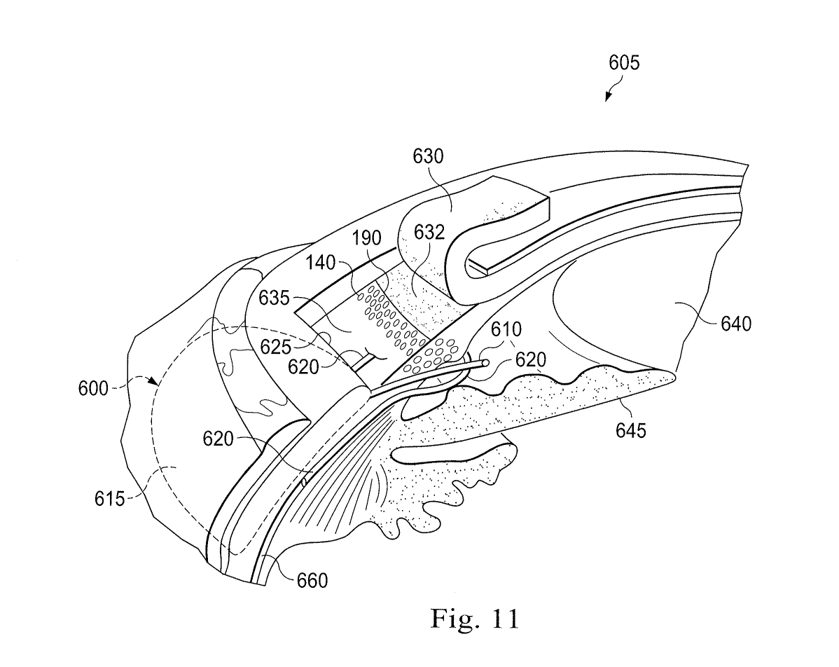

FIGS. 11-15 illustrate exemplary methods of implanting an exemplary drainage device 600 in an eye 605. The drainage device 600 includes an inlet tube 610, a flow system or body 615, and an outlet tube 620. As shown in FIG. 11, the drainage device 600 is implanted into a subconjunctival space 625 (i.e., between a conjunctiva 630, which covers the cornea 632 and the sclera 635). The inlet tube 610 allows aqueous humor to flow from an anterior chamber 640 to the flow system 615 positioned within the subconjunctival space 625. In the pictured embodiment, the inlet tube 610 from the flow system 615 located in the subconjunctival space 625 enters the anterior chamber 640 through the corneoscleral limbus 190 and parallel to the iris 645. The outlet tube 620 allows aqueous humor to drain from the flow system 615 into a suprachoroidal space 660. The outlet tube 620 extends from the flow system 615 in the subconjunctival space 625 to the anterior chamber 640 through the corneoscleral limbus 190 and parallel to the iris 645 to enter the anterior chamber 640 before turning back on itself to enter the suprachoroidal space 660.

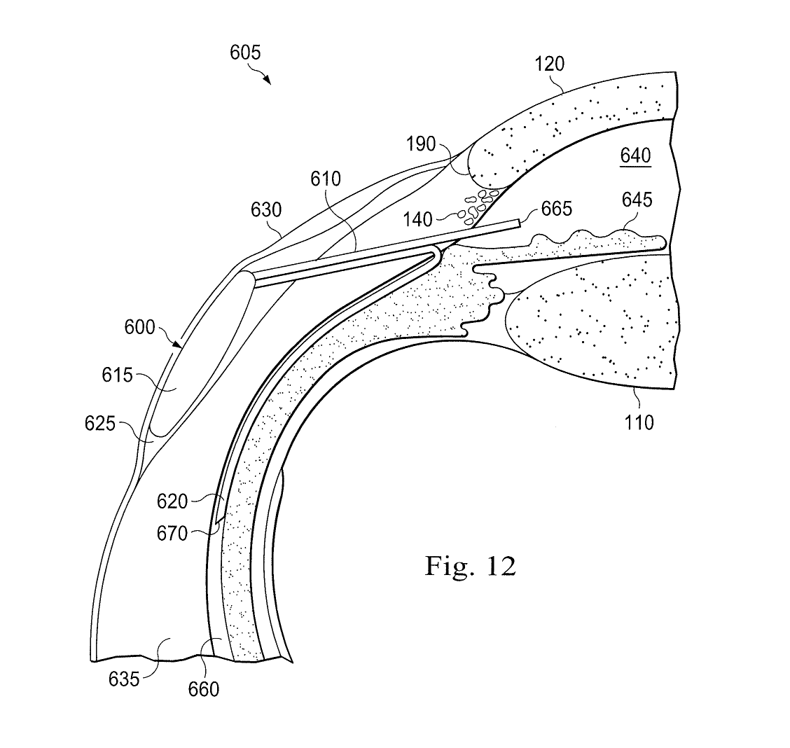

FIG. 12 illustrates a side view of the drainage device 600 implanted within the subconjunctival space 625 of the eye 605. As also shown in FIG. 11, the inlet tube 610 extends from the anterior chamber 640, parallel to the iris 645, through the corneoscleral limbus 190, and into the subconjunctival space 625 where it joins the flow system 615. The outlet tube 620 extends from the flow system 615 in the subconjunctival space 625, parallel to the iris 645, and through the corneoscleral limbus 190 to enter the anterior chamber 640 before turning back on itself to enter the suprachoroidal space 660. In some instances, the inlet tube 610 and the outlet tube 620 include sufficiently strong or sharp distal ends 665, 670, respectively, to tunnel through the sclera 635 and the trabecular meshwork 140.

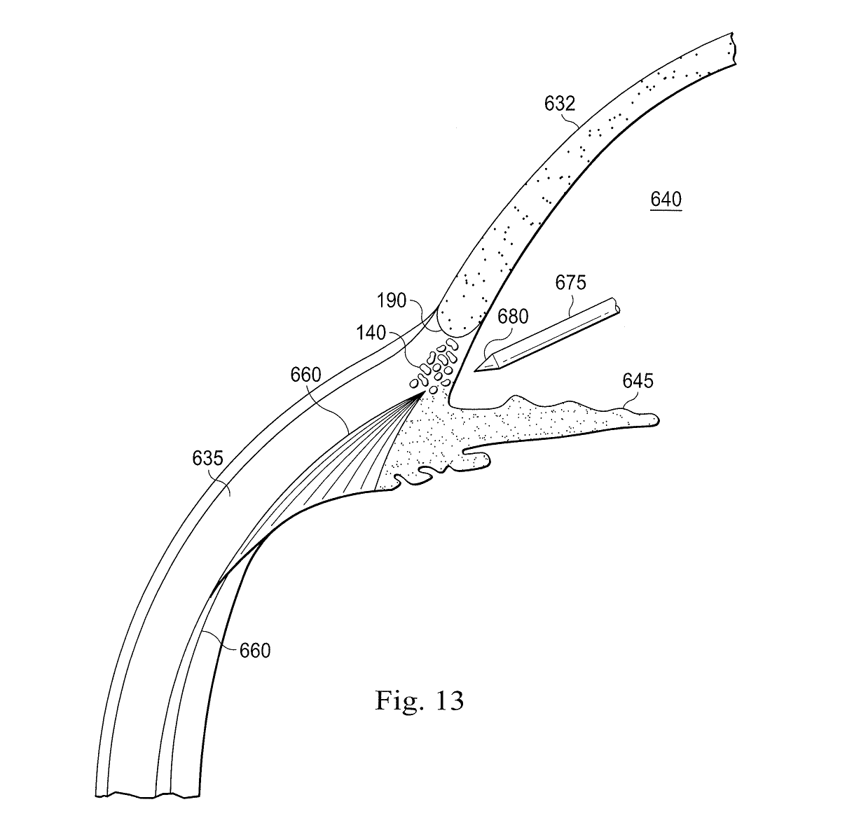

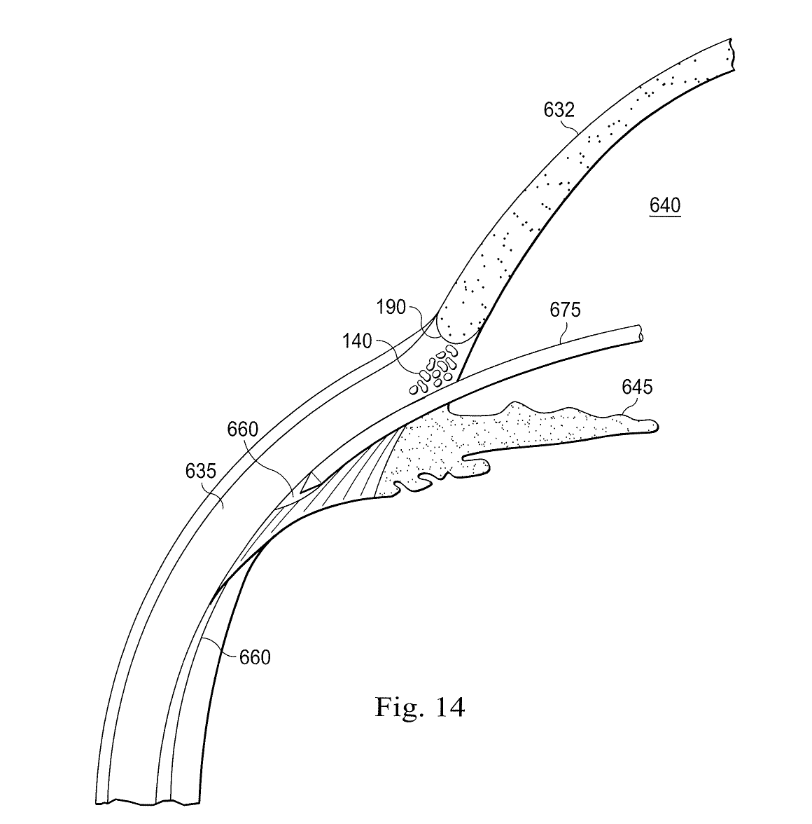

However, in some instances, a surgeon may use one or more surgical instruments to create a pathway for the outlet tube (and/or the inlet tube) prior to (or during) implantation of the drainage device 600. In particular, the surgeon may employ this technique when the inlet and outlet tubes 610, 620 include blunt or rounded atraumatic ends 665, 670, respectively. As shown in FIG. 13, using an ab interno approach, the surgeon may insert a tunneling instrument 675 through the cornea 632 into the anterior chamber 640. In the pictured embodiment, the tunneling instrument 675 includes a distal tip 680. The distal tip 680 is sufficiently sharp to penetrate the cornea 632 through the corneoscleral limbus 190 to enter the suprachoroidal space 660. After inserting the tunneling instrument 675 into the anterior chamber 640, the surgeon may advance the tunneling instrument 675 through the anterior chamber toward the trabecular meshwork 140 and the suprachoroidal space 660. In some instances, as shown in FIG. 14, the surgeon advances the tunneling instrument 675 toward and above the corneoscleral limbus 190 before entering the suprachoroidal space 660 (e.g., to create a pathway for the outlet tube 620) and/or the subconjunctival space (e.g., to create a pathway for the inlet tube 610 and/or the outlet tube 620). In some embodiments, the tunneling instrument 675 may be steerable, articulating, or shapeable in a manner that facilitates the proper approach of the tunneling instrument 675 toward the desired ocular tissues.

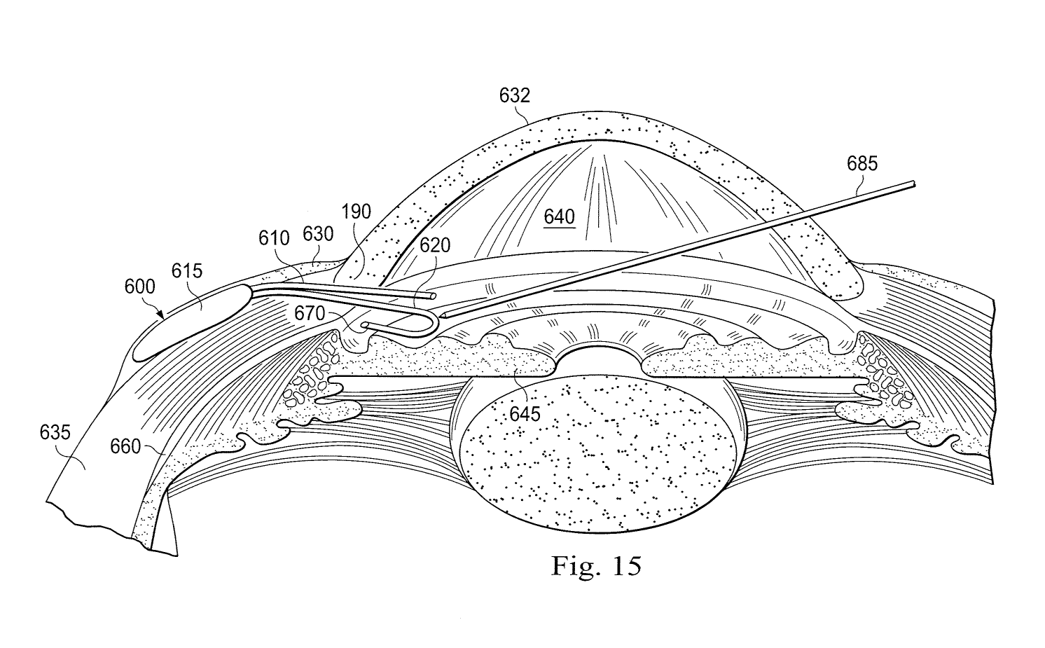

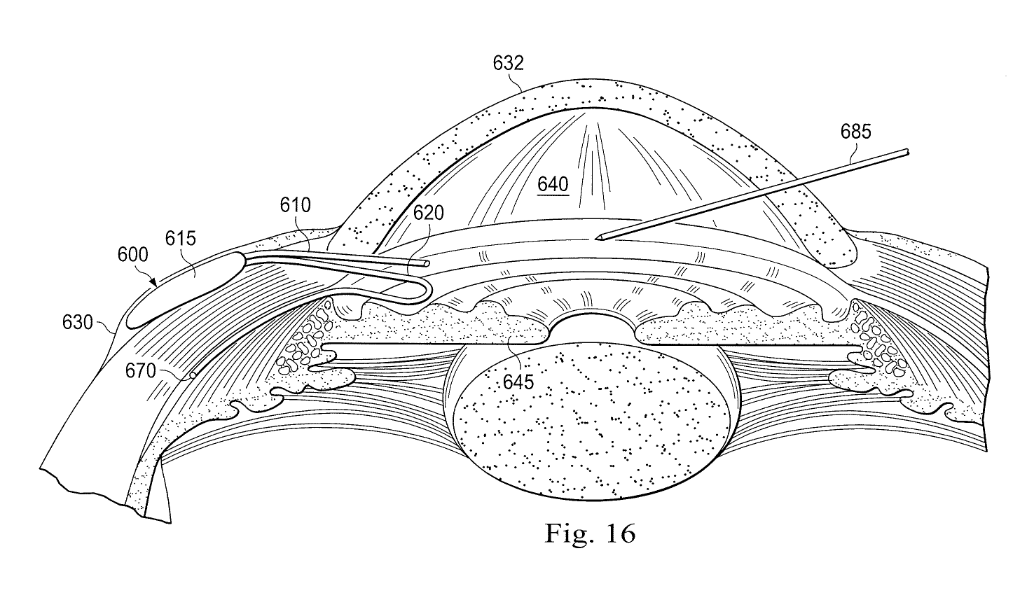

As illustrated in FIG. 15, after inserting the drainage device 600 into the subconjunctival space and withdrawing the tunneling instrument 675 from the anterior chamber 640, the surgeon may insert a guiding instrument 685 through the cornea 632 to steer (e.g., by grasping or nudging) the distal end 670 of the outlet tube 620 toward the suprachoroidal space 660. FIG. 16 illustrates the outlet tube 620 positioned within the suprachoroidal space 660. As shown in FIG. 16, after the outlet tube 620 is properly positioned within the suprachoroidal space 660, the surgeon may withdraw the guiding instrument 685 from the anterior chamber 640 through the cornea 632.

FIGS. 4 and 17-19 illustrate an exemplary method of implanting the drainage implant 300 in the eye 10. As described above, the implant 300 includes the inlet tube 325, the flow system or body 315, and the outlet tube 330. As shown in FIG. 4, the body or flow system 315 of the implant 300 is positioned within the eye in the subconjunctival space 345 between the conjunctiva 202 and the sclera 180. The inlet tube 325 allows aqueous humor to flow from the anterior chamber 170 to the flow system 315 positioned within the subconjunctival space 345. The inlet tube 325 and the outlet tube 330 are coupled to the flow system 315 at the location of the subconjunctival space 345. In the pictured embodiment, the inlet tube 325 extends from the anterior chamber 170 (from a position above the iris 130), through the trabecular meshwork 150 at the region of a scleral spur 682, and into the subconjunctival space 345 where it joins the flow system 315. The inlet tube 325 allows aqueous humor to exit the anterior chamber 170 and enter the flow system 315. The outlet tube 330 extends from the flow system 315 in the subconjunctival space 345 through the sclera 180 before entering the suprachoroidal space 200. The outlet tube 330 allows aqueous humor to drain from the flow system 315 into the suprachoroidal space 200.

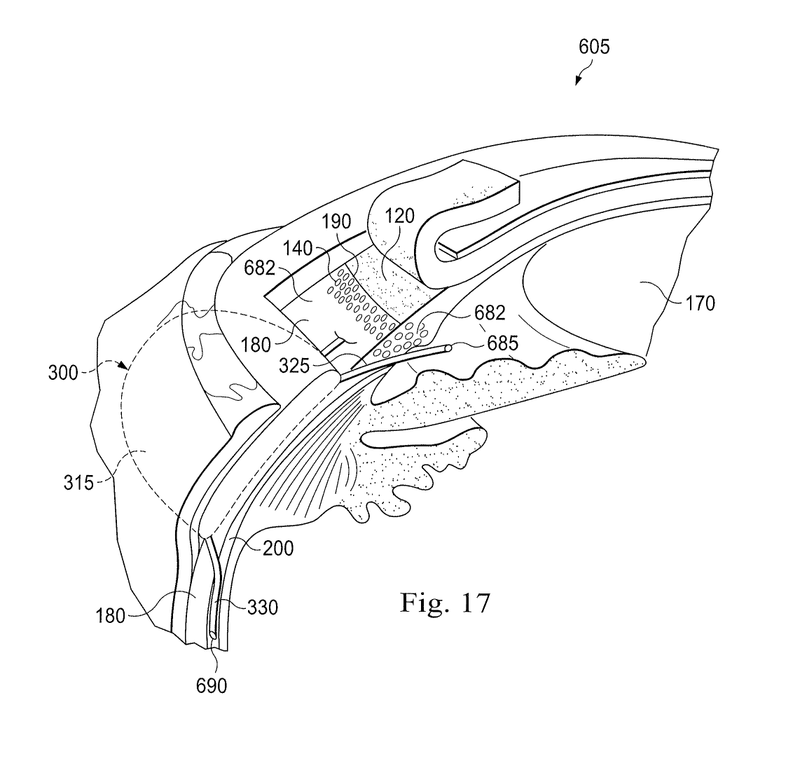

FIG. 4 illustrates a side view of the drainage implant 300 implanted within the subconjunctival space 345 of the eye 10. As also shown in FIG. 17, the inlet tube 325 extends from the anterior chamber 170, above the scleral spur 682, and into the subconjunctival space 345 where it joins the flow system 315. The outlet tube 330, unlike the outlet tube 620 shown in FIGS. 11-15, extends from the flow system 315 in the subconjunctival space 345 through the sclera 180 to directly enter the suprachoroidal space 200 (i.e., without first accessing the anterior chamber 170). In some instances, the inlet tube 325 and the outlet tube 330 include sufficiently strong or sharp distal ends 685, 690, respectively, to tunnel through the sclera 180.

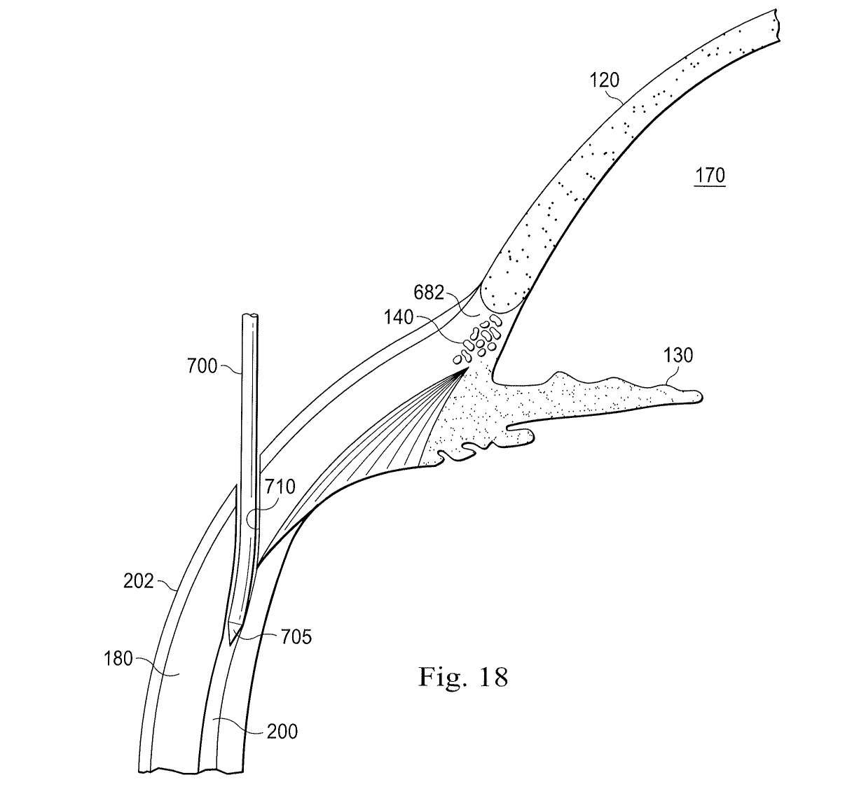

However, in some instances, a surgeon may use one or more surgical instruments to create a pathway for the outlet tube (and/or the inlet tube) prior to (or during) implantation of the drainage implant 300. In particular, the surgeon may employ this technique when the inlet and outlet tubes 325, 330 include blunt or rounded atraumatic ends 685, 690, respectively. As shown in FIG. 18 (utilizing a fornix space or limbus based conjunctival incision, with conjunctival tenting not shown), using an ab externo approach, the surgeon may insert a tunneling instrument 700 through the conjunctiva 202 and the sclera 180 to create a path or channel accessing the suprachoroidal space 200. In the pictured embodiment, the tunneling instrument 700 includes a distal tip 705. The distal tip 705 is sufficiently sharp to penetrate the conjunctiva 202 and the sclera 180 to enter the suprachoroidal space 200. After passing the tunneling instrument 700 through the sclera 180 to create a path or tunnel 710, the surgeon may advance the tunneling instrument 700 a desired distance into the suprachoroidal space 200. In some embodiments, the tunneling instrument 700 may be steerable, articulating, or shapeable in a manner that facilitates the proper approach of the tunneling instrument 700 toward the desired ocular tissues.

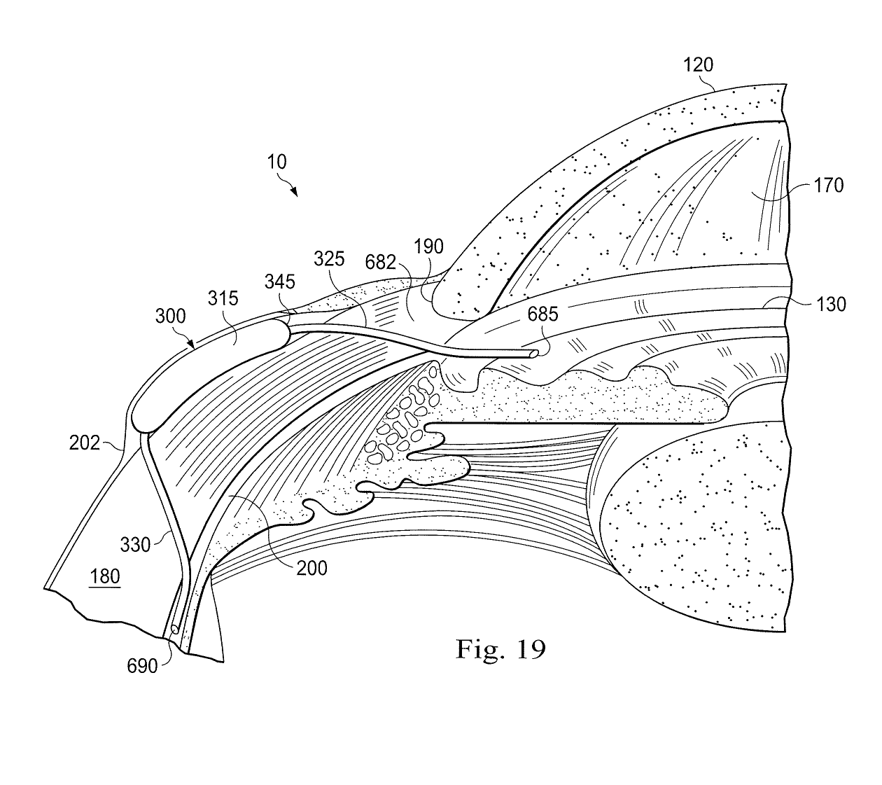

As illustrated in FIG. 19, after withdrawing the tunneling instrument 700 from the anterior chamber 170, the surgeon may position the drainage implant 300 into the subconjunctival space 345 and guide the outlet tube 330 through the path 710 in the sclera 180 into the suprachoroidal space 200. FIG. 19 illustrates the distal end 690 of the outlet tube 330 positioned within the suprachoroidal space 200, and the distal end 685 of the inlet tube 325 positioned within the anterior chamber 170.

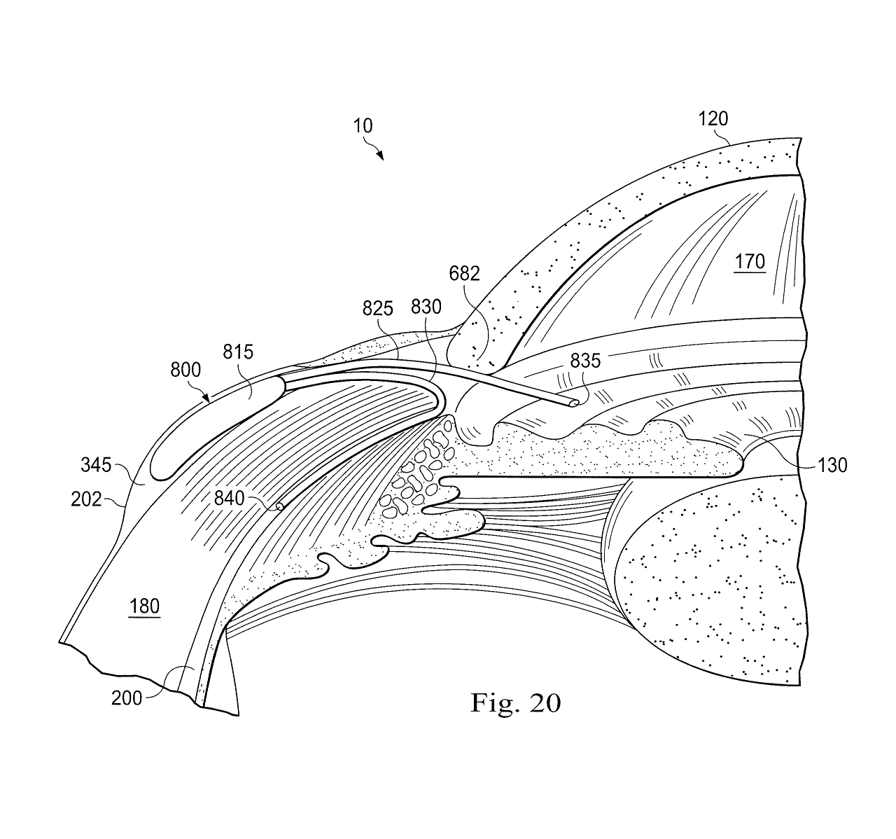

FIG. 20 illustrates an exemplary method of implanting the drainage implant 800 in the eye 10. The implant 800 is substantially similar to the drainage implant 300 except for the differences noted herein. The implant 800 includes a flow system or body 815, an inlet tube 825, and the outlet tube 830. The flow system 815 of the implant 800 is positioned within the eye in the subconjunctival space 345 between the conjunctiva 202 and the sclera 180. The inlet tube 825 allows aqueous humor to flow from the anterior chamber 170 to the flow system 815 positioned within the subconjunctival space 345. The inlet tube 825 and the outlet tube 830 are coupled to the flow system 815 at the location of the subconjunctival space 345. In the pictured embodiment, the inlet tube 825 extends from the anterior chamber 170 (from a position above the iris 130), through the trabecular meshwork 150 at the region of a scleral spur 682, and into the subconjunctival space 345 where it joins the flow system 815. The inlet tube 825 allows aqueous humor to exit the anterior chamber 170 and enter the flow system 815. The outlet tube 830, unlike the outlet tube 620 shown in FIGS. 11-15, extends from the flow system 315 in the subconjunctival space 345 through the sclera 180 to enter the suprachoroidal space 200 (i.e., without first accessing the anterior chamber 170). In particular, the outlet tube 830 extends from the flow system 815 in the subconjunctival space 345 through the sclera 180 (either at or posterior to the scleral spur 682) and curves or turns back on itself to enter the suprachoroidal space 200. The outlet tube 830 allows aqueous humor to drain from the flow system 815 into the suprachoroidal space 200. In some instances, the inlet tube 825 and the outlet tube 830 include sufficiently strong or sharp distal ends 835, 840, respectively, to tunnel through the sclera 180.

However, in some instances, a surgeon may use one or more surgical instruments to create a pathway for the outlet tube (and/or the inlet tube) prior to (or during) implantation of the drainage implant 800. In particular, the surgeon may employ this technique when the inlet and outlet tubes 825, 830 include blunt or rounded atraumatic ends 835, 840, respectively. Similar to the method shown in FIG. 18, using an ab externo approach, the surgeon may insert a tunneling instrument 700 through the conjunctiva 202 and the sclera 180 to create a curved path or channel accessing the suprachoroidal space 200.

Embodiments in accordance with the present disclosure provide a fluid drainage device which utilizes an adjustable smart valve, a passive valve, or a pump to drain aqueous humor from the anterior chamber to a drainage site remote from the subconjunctiva. In particular, embodiments in accordance with the present disclosure provide a fluid drainage device which utilizes an adjustable smart valve, a passive valve, or a pump to drain aqueous humor from the anterior chamber to the suprachoroidal space via an outlet tube disposed in the suprachoroidal space. Allowing the aqueous humor to drain into a site remote from the subconjunctiva minimizes the problems that may be associated with subconjunctival drainage, including subconjunctival bleb failure due to fibrosis, conjunctival leakage, infections, and/or endophthalmitis.

Persons of ordinary skill in the art will appreciate that the embodiments encompassed by the present disclosure are not limited to the particular exemplary embodiments described above. In that regard, although illustrative embodiments have been shown and described, a wide range of modification, change, and substitution is contemplated in the foregoing disclosure. It is understood that such variations may be made to the foregoing without departing from the scope of the present disclosure. Accordingly, it is appropriate that the appended claims be construed broadly and in a manner consistent with the present disclosure.

We claim:

1. A treatment device for the drainage of fluid within an eye of a patient, comprising:
a drainage tube having a lumen and comprising an inlet tube portion and an outlet tube portion, the drainage tube configured to convey aqueous humor through the lumen from an anterior chamber of the eye to a suprachoroidal space of the eye; and
a flow system in fluid communication with the drainage tube, the flow system configured to control intraocular pressure by throttling flow rates of the aqueous humor through the drainage tube,
wherein the inlet tube portion is arranged to extend from the anterior chamber to the flow system, and the outlet tube portion is flexible to conform to the curvature of the suprachoroidal space and is arranged to extend from the valve system to the suprachoroidal space.

2. The treatment device of claim 1, wherein the flow system is arranged to be positioned within the subconjunctival space.

3. The treatment device of claim 1, wherein the outlet tube portion includes a proximal end coupled to the flow system and a distal end arranged to be positioned within the suprachoroidal space, and an outlet tube lumen extending from the proximal end to the distal end.

4. The treatment device of claim 3, wherein the outlet tube portion is arranged to extend from the flow system into the anterior chamber and the distal end of the outlet tube portion is arranged to be in communication with the suprachoroidal space.

5. The treatment device of claim 4, wherein the outlet tube portion arranged to extend from the flow system through a sclera into the anterior chamber and curve back on itself to enter the suprachoroidal space.

6. The treatment device of claim 5, wherein the outlet tube portion arranged to extend from the flow system through a corneoscleral limbus, enter the anterior chamber, and curve back on itself to enter the suprachoroidal space.

7. The treatment device of claim 3, wherein the outlet tube portion is arranged to extend from the flow system through a sclera and the distal end is arranged to communicate with the suprachoroidal space.

8. The treatment device of claim 7, wherein the outlet tube portion is arranged to extend from the flow system through the sclera and curve back on itself within the sclera to enter the suprachoroidal space.

9. The treatment device of claim 1, wherein the flow system is actuatable in response to pressure differentials and is configured to control flow rates of the aqueous humor along the drainage tube by shifting in response to pressure differentials between the anterior chamber of the eye, the suprachoroidal space, and atmospheric pressure acting on the flow system or any combination thereof.

10. The treatment device of claim 9, wherein the flow system includes a pressure-driven valve system.

11. The treatment device of claim 10, wherein the flow system includes an electrically-driven pump system in fluid communication with the drainage tube and the pressure-driven valve system, the pump system being arranged to selectively control the flow of aqueous humor through the drainage tube from the anterior chamber into the suprachoroidal space.

12. The treatment device of claim 10, wherein the pressure-driven valve system includes at least one flow control membrane.

13. The treatment device of claim 3, wherein the distal end of the outlet tube portion includes blunt edges.

14. The treatment device of claim 3, wherein the distal end of the outlet tube portion includes sharp edges configured to pierce ocular tissue.

15. The treatment device of claim 1, wherein the drainage tube includes markers configured to indicate the position of the inlet tube portion and outlet tube portion within the eye.

16. The treatment device of claim 15, wherein the markers comprise radiopaque markers.

17. The treatment device of claim 3, wherein the lumen of the outlet tube portion includes a substantially uniform luminal diameter from the proximal end to the distal end.

18. The treatment device of claim 3, wherein the lumen of the outlet tube portion includes a first luminal diameter at the proximal end and a second luminal diameter at the distal end, the first luminal diameter being less than the second luminal diameter.

19. The treatment device of claim 3, wherein the outlet tube portion includes a plurality of apertures in communication with the lumen arranged so that aqueous humor may exit the outlet tube portion through the plurality of apertures.

20. The treatment device of claim 3, wherein the outlet tube portion includes a proximal aperture at the proximal end and a distal aperture at the distal end, wherein the outlet tube lumen extends from the proximal aperture to the distal aperture.

21. The treatment device of claim 3, wherein the outlet tube portion includes a plurality of drainage features configured to facilitate the passage of aqueous humor through the lumen from the proximal end to the distal end.

22. The treatment device of claim 3, wherein the outlet tube portion includes retention features configured to anchor the distal end within the suprachoroidal space.

23. A treatment device for the drainage of fluid within an eye of a patient, comprising:
an implant positionable in a subconjunctival space of the eye; and
an outlet tube portion having a lumen extending from a proximal aperture to a distal aperture, the outlet tube portion configured to convey aqueous humor through the lumen from the implant to a suprachoroidal space of the eye,
wherein the outlet tube portion is arranged to extend from the implant through a sclera of the eye into the anterior chamber and is arranged to curve back on itself to enter the suprachoroidal space, and
wherein the proximal aperture is in fluid communication with the implant, and the distal aperture is in communication with the suprachoroidal space.

24. The treatment device of claim 23, wherein the outlet tube portion is arranged to extend from the implant through a corneoscleral limbus, enter the anterior chamber, and curve back on itself to enter the suprachoroidal space.

25. The treatment device of claim 23, wherein the outlet tube portion arranged to extend from the implant through a sclera.

26. The treatment device of claim 25, wherein the outlet tube portion arranged to extend from the implant through the sclera and curve back on itself within the sclera to enter the suprachoroidal space.

27. The treatment device of claim 23, wherein the distal aperture of the outlet tube portion includes blunt edges.

28. The treatment device of claim 23, wherein the distal aperture of the outlet tube portion includes sharp edges configured to pierce ocular tissue.

29. The treatment device of claim 23, wherein the outlet tube portion includes markers configured to indicate the position of the distal aperture within the eye.

30. The treatment device of claim 23, wherein the lumen of the outlet tube portion includes a substantially uniform luminal diameter from the proximal aperture to the distal aperture.

31. The treatment device of claim 23, wherein the lumen of the outlet tube portion includes a first luminal diameter at the proximal aperture and a second luminal diameter at the distal aperture, the first luminal diameter being less than the second luminal diameter.

32. The treatment device of claim 23, wherein the outlet tube portion includes a plurality of apertures in communication with the lumen, wherein aqueous humor may exit the outlet tube through the plurality of apertures.

33. The treatment device of claim 23, wherein the outlet tube portion includes a plurality of drainage features configured to facilitate the passage of aqueous humor through the lumen from the proximal aperture to the distal aperture.

34. The treatment device of claim 23, wherein the outlet tube portion includes retention features configured to anchor the distal aperture within the suprachoroidal space.

35. A method of implanting a treatment device into an eye of a patient, comprising:
inserting a drainage device including a flow system, an inlet tube, and an outlet tube into a subconjunctival space, wherein the inlet tube includes a first proximal end coupled to the flow system in the subconjunctival space, and wherein the outlet tube includes a second proximal end coupled to the flow system in the subconjunctival space;
passing a first distal end of the inlet tube through a sclera into an anterior chamber; and
passing a second distal end of the outlet tube through the sclera into the suprachoroidal space.

36. The method of claim 35, wherein passing a second distal end of the outlet tube through the sclera into the suprachoroidal space comprises turning the outlet tube back on itself within the sclera before the second distal end enters the suprachoroidal space.

37. The method of claim 35, wherein passing a second distal end of the outlet tube through the sclera into the suprachoroidal space comprises passing the second distal end of the outlet tube into the anterior chamber and turning the outlet tube back on itself within the anterior chamber before the second distal end enters the suprachoroidal space.

38. The method of claim 37, wherein passing a second distal end of the outlet tube through the sclera into the suprachoroidal space comprises passing the second distal end of the outlet tube into the anterior chamber and turning the outlet tube back on itself within the anterior chamber to pass adjacent to a scleral spur before entering the suprachoroidal space.

39. The method of claim 35, further including using a tunneling instrument to create a passageway for the second distal end of the outlet tube from the subconjunctival space through the sclera to the suprachoroidal space.

40. The method of claim 40, wherein using a tunneling instrument to create a passageway for the second distal end of the outlet tube from the subconjunctival space through the sclera to the suprachoroidal space comprises inserting the tunneling instrument through a corneoscleral limbus into the anterior chamber and creating a passageway from the anterior chamber adjacent to a scleral spur into the suprachoroidal space.

41. The method of claim 39, wherein using a tunneling instrument to create a passageway for the second distal end of the outlet tube from the subconjunctival space through the sclera to the suprachoroidal space comprises inserting the tunneling instrument through the sclera into the suprachoroidal space.

42. The method of claim 37, further including guiding the second distal end of the outlet tube from the anterior chamber through the sclera to the suprachoroidal space with a guiding instrument.

43. The method of claim 42, wherein guiding the second distal end of the outlet tube from the anterior chamber through the sclera to the suprachoroidal space comprises inserting the guiding instrument through a cornea into the anterior chamber and manipulating the outlet tube in the anterior chamber.
